(19) United States
(12) Patent Application Publication
(10) Pub. No.: US 2007/0135518 A1
(43) Pub. Date: Jun. 14, 2007

(54) Use of low-dose ladostigil for neuroprotection

(76) Inventors: Marta Weinstock-Rosin, Jerusalem (IL); Shai Shoham, Jerusalem (IL)

Correspondence Address: COOPER & DUNHAM, LLP, 1185 AVENUE OF THE AMERICAS, NEW YORK, NY, 10036, US

(21) Appl. No.: 11/635,922

(22) Filed: Dec. 8, 2006

Related U.S. Application Data
(60) Provisional application No. 60/792,410, filed on Apr. 17, 2006.
(60) Provisional application No. 60/748,748, filed on Dec. 9, 2005.

(51) Int. Cl. A61K 31/325

(52) U.S. Cl. 514/485

(57) ABSTRACT

The subject invention provides a method of preventing a neurodegenerative disease in a subject or oxidative stress in the brain of a subject, comprising administering to the subject a less than cholinesterase-inhibitory amount or a less than monoamine oxidase-inhibitory amount of R(+)-6-(N-methyl,N-ethyl-carbamoyloxy)-N′-propargyl-1-aminoindan or a salt thereof effective to prevent the neurodegenerative disease or oxidative stress in the subject.

This application claims benefit of U.S. Provisional Application No. 60/792,410, filed Apr. 17, 2006 and U.S. Provisional Application No. 60/748,748, filed Dec. 9, 2005 the contents of which are hereby incorporated by reference.

Throughout this application various publications, published patent applications, and patents are referenced. The disclosures of these documents in their entireties are hereby incorporated by reference into this application in order to more fully describe the state of the art to which this invention pertains.

### BACKGROUND OF THE INVENTION

Oxidative stress has been proposed as a pathogenic mechanism in Alzheimer's disease (AD) (P. F. Good et al., Am. J. Pathol. (1996) 149:21). Oxidative stress may also contribute to neuronal degeneration and death in disorders ranging from ischemic stroke to Alzheimer's and Parkinson's to age related macular degeneration to amyotrophic lateral sclerosis (M. P. Mattson et al., J. Neurosci. Res. (1997) 49:681), disorders in which nitric oxide, via peroxynitrite, plays a key role.

Several strategies for conferring neuroprotection have been developed which target the complex neurochemical processes which follow neuronal malfunction. Older approaches (reviewed by N. G. Wahlgren, in R. Green, “International Review of Neurobiology: Neuroprotective Agents and Cerebral Ischemia”, Vol. 40, Academic Press, 1997) including closure of calcium channels (with calcium antagonists), inhibition of glutamate release, and antagonism to NMDA and GABA agonism have not led to any remarkable treatments. With the recent emphasis on the role of reactive oxygen species (ROS) and of the nitrogen oxyanion species, the focus of possible treatments has now shifted to antioxidant and free radical scavengers (K. Hensley et al., in “Neuroinflammation: mechanisms and management” (Ed: P. L. Wood), Humana Press Inc., 1997). R(+)-6-(N-methyl,N-ethyl-carbamoyloxy)-N′-propargyl-1-aminoindan, also known as (3R)-3-(prop-2-ynylamino)-2,3,-dihydro-1H-inden-5-yl ethylmethylcarbamate, is disclosed in PCT Application Publication No. WO98/27055 (U.S. Pat. No. 6,303,650, issued Oct. 16, 2001 to Chorev), the entire contents of which are incorporated by reference. This compound has been given the nonproprietary name ladostigil.

Ladostigil has been shown to inhibit acetylcholinesterase (ChE) and monoamine oxidase selectively in the brain (M. Weinstock et al., TV3326, a novel neuroprotective drug with cholinesterase and monoamine oxidase inhibitory activities for the treatment of Alzheimer's disease, J. Neural Transm. Suppl. (2000) 60:157-69). As such, ladostigil has been proposed for treatment of depression, Attention Deficit Disorder (ADD), Attention Deficit and Hyperactivity Disorder (ADHD), Tourette's Syndrome, Alzheimer's Disease and other dementias, neurotraumatic disorders and memory disorders in humans (see, e.g., U.S. Pat. No. 6,538,025, issued Mar. 25, 2003 to Chorev et al.). The following dosing of ladostigil has been disclosed: chronic administration of 52 mg/kg to treat comorbidity of dementia with Parkinsonism in a rat model (Y. Sagi, The neurochemical and behavioural effects of the novel cholinesterase-monoamine oxidase inhibitor, ladostigil, in response to L-dopa and L-tryptophan, in rats, Br. J. Pharmacol. (2005) 146(4):553-60); chronic administration of 17 mg/kg to treat hyperanxiety and depressive-like behaviour in a rat model (T. Poltyrev et al., Effect of chronic treatment with ladostigil (TV-3326) on anxiogenic and depressive-like behaviour and on activity of the hypothalamic-pituitary-adrenal axis in male and female prenatally stressed rats, Psychopharmacology (2005) 181(1):118-25); and chronic administration of 26 mg/kg to show brain selective MAO inhibition (M. Weinstock, Limited potentiation of blood pressure response to oral tyramine by brain-selective monoamine oxidase A-B inhibitor, TV-3326 in conscious rabbits, Neuropharmacology (2002) 43(6) :999-1005, and M. Weinstock, Effect of TV3326, a novel monoamine-oxidase cholinesterase inhibitor, in rat models of anxiety and depression, Psychopharmacology (2002) 160(3):318-24, respectively). Ladostigil has also been shown to suppress apoptosis induced by the peroxynitrite-generating agent N-morpholino sydnonimine (Sin-1) in cultured dopaminergic neuroblastoma SH-SY5Y cells (Maruyama et al., Anti-apoptotic action of anti-Alzheimer drug, TV3326 [(N-propargyl)-(3R)-aminoindant-5-yl]-ethyl methyl carbamate, a novel cholinesterase-monoamine oxidase inhibitor, Neuroscience Letters (2003) 341:233-236).

MAO inhibitors (MAOIs) are known to have many contraindications, and are associated with high incidences of hypertensive crises (Physician's Desk Reference, 59^{th} Ed. (2005) pgs. 1583-4).

ChE inhibition is known to lower body temperature. (Gordon, 1994) By stimulating muscarinic receptors in the pre-optic area of the hypothalamus, heat loss is promoted through peripheral vasodilatation. It has been shown that the fall in body temperature is proportional to the degree of brain ChE inhibition.

Disclosed herein is that ladostigil can be used to reduce the neurodegenerative effects of oxidative nitrative stress at dosage levels that do not cause the noted side effects related to inhibition of monoamine oxidase (MAO) or cholinesterase (ChE).

### SUMMARY OF THE INVENTION

The subject invention provides a method of preventing the appearance of symptoms of a neurodegenerative disease in a subject predisposed to the neurodegenerative disease, or of slowing the progression of an early stage neurodegenerative disease to a more advanced stage of the neurodegenerative disease in an afflicted subject, comprising administering to the subject an amount of R(+)-6-(N-methyl,N-ethyl-carbamoyloxy)-N′-propargyl-1-aminoindan or a salt thereof effective to prevent the appearance of symptoms of the disease or to slow the progression of the disease.

The subject invention also provides a method of reducing oxidative stress in the brain of a subject afflicted with oxidative stress, comprising administering to the subject a less than cholinesterase-inhibitory amount or a less than monoamine oxidase-inhibitory amount of R(+)-6-(N-methyl,N-ethyl-carbamoyloxy)-N′-propargyl-1-aminoindan or a salt thereof effective to reduce oxidative stress in the brain of the subject.

The subject invention also provides a method of treating a subject afflicted with mild cognitive impairment comprising administering to the subject an amount of R(+)-6-(N-methyl,N-ethyl-carbamoyloxy)-N′-propargyl-1-aminoindan or a salt thereof effective to treat the subject.

The subject invention also provides a pharmaceutical composition in unit dosage form comprising R(+)-6-(N-methyl,N-ethyl-carbamoyloxy)-N′-propargyl-1-aminoindan in an amount of up to 25 mg, or a corresponding amount of the salt thereof, and a pharmaceutically acceptable carrier.

### BBIEF DESCRIPTION OF THE FIGURES

FIG. 1 Effect of ladostigil on nitrotyrosine immunoreactivity in astrocytes of 3 hippocampal regions in the CA1 area one week after a bilateral intracerebroventricular (icv) injection of STZ. An * indicates that the result is significantly different from CSF and STZ+ladostigil, P<0.01.

FIG. 2 a) Diagram of brain section showing the three zones in the vicinity of the cannula penetration site with differential microglia and astrocyte activation 7 days after icv STZ.
b) Microglia: y zones i) microglia have round shaped soma devoid of processes; ii) microglia with polymorphic shaped soma and a variety of fibrous processes; iii) quiescent microglia.
c) Astrocytes: zones i) astrocyte-free; ii) astrocytes with elongated processes but no soma; iii) reactive astrocytes with thickened processes and filled soma.
Area box in b) and c) is shown in higher magnification in d) and e) respectively. Calibration bar=100 μm in b) and c) and 20 μm in d) and e).

FIG. 3 Quantification of activation of microglia and astrocytes in zones shown in FIG. 2.
a) Counts of microglia subtypes in zones i) and ii).
b) % of wound cross sectional area occupied by zones i), ii) and iii).
In zone (i) the area free of astrocytes is greater in STZ-injected rats. Significantly different from other groups, **P<0.01, *P<0.05.

FIG. 4 Quantification of astrocytes with NT expression in cannula penetration site and in three other brain areas bordering the lateral ventricles.
CP=cannula penetration site; CC=corpus callosum; LS=lateral septum; MS=medial septum HP=hippocampal CA1 area. Significantly different from other groups, *P<0.01.

FIG. 5 Confocal picture of NT expression in reactive astrocytes in hippocampus, including localization of NT immunoreactivity in activated astrocytes.

FIG. 6 Effect of ladostigil on spatial memory deficit in the Morris water maze induced by icv STZ. a) trial 1; b) trial 2.
Significantly different from other groups *P<0.05.

FIG. 7 Comparison of spatial learning in young and old rats in the Morris water maze (MWM). Prevention by 1 mg/kg of ladostigil of learning deficits in old rats; a) shows escape latency on each of two daily trials. Note much larger day-day variability in escape learning in aged untreated rats that is prevented by chronic treatment with ladostigil; b) average escape latency for two daily trials.

### DETAILED DESCRIPTION

The subject invention provides a method of preventing the appearance of symptoms of a neurodegenerative disease in a subject predisposed to the neurodegenerative disease, or of slowing the progression of an early stage neurodegenerative disease to a more advanced stage of the neurodegenerative disease in an afflicted subject, comprising administering to the subject an amount of R(+)-6-(N-methyl,N-ethyl-carbamoyloxy)-N′-propargyl-1-aminoindan or a salt thereof effective to prevent the appearance of symptoms of the disease or to slow the progression of the disease.

In an embodiment, the effective amount administered to the subject is a less than cholinesterase-inhibitory amount or a less than monoamine oxidase-inhibitory amount of R(+)-6-(N-methyl,N-ethyl-carbamoyloxy)-N′-propargyl-1-aminoindan or a salt thereof.

In an embodiment, the method comprises administration to the subject a less than cholinesterase-inhibitory amount and a less than monoamine oxidase-inhibitory amount of R(+)-6-(N-methyl,N-ethyl-carbamoyloxy)-N′-propargyl-1-aminoindan or a salt thereof.

In another embodiment, the method comprises administration of a salt of R(+)-6-(N-methyl,N-ethyl-carbamoyloxy)-N′-propargyl-1-aminoindan.

In another embodiment of the method, the salt of R(+)-6-(N-methyl,N-ethyl-carbamoyloxy)-N′-propargyl-1-aminoindan is the tartrate salt.

In yet another embodiment of the method, the administration is acute administration.

In yet another embodiment of the method, the subject is a human.

In yet another embodiment of the method with acute administration to humans, the less than monoamine oxidase-inhibitory amount of R(+)-6-(N-methyl,N-ethyl-carbamoyloxy)-N′-propargyl-1-aminoindan is no more than 1.7 mg/kg/day of the subject, or a corresponding amount of the salt thereof.

In yet another embodiment of the method with acute administration to humans, the salt is the tartrate salt and the corresponding amount of the salt is no more than 2.1 mg/kg/day of the subject.

In yet another embodiment of the method with acute administration to humans, the less than cholinesterase-inhibitory amount of R(+)-6-(N-methyl,N-ethyl-carbamoyloxy)-N′-propargyl-1-aminoindan is no more than 2.3 mg/kg/day of the subject, or a corresponding amount of the salt thereof.

In yet another embodiment of the method with acute administration to humans, the salt is the tartrate salt and the corresponding amount of the salt is no more than 2.9 mg/kg/day of the subject.

In a further embodiment of the method, the administration is chronic administration.

In a further embodiment of the method, the subject is a human.

In yet a further embodiment of the method with humans, the amount of R(+)-6-(N-methyl,N-ethyl-carbamoyloxy)-N′-propargyl-1-aminoindan is no more than 0.37 mg/kg/day of the subject, or a corresponding amount of the salt thereof.

In yet a further embodiment of the method with to humans, the salt is the tartrate salt and the corresponding amount of the salt is no more than 0.46 mg/kg/day of the subject.

In yet a further embodiment of the method with to humans, the amount of R(+)-6-(N-methyl,N-ethyl-carbamoyloxy)-N′-propargyl-1-aminoindan is no more than 2.3 mg/kg/day of the subject, or a corresponding amount of the salt thereof.

In yet a further embodiment of the method with to humans, the salt is the tartrate salt and the corresponding amount of the salt is no more than 2.9 mg/kg/day of the subject.

In another embodiment of the method, the amount of R(+)-6-(N-methyl,N-ethyl-carbamoyloxy)-N′-propargyl-1-aminoindan or a salt thereof administered is 10 mg/day-25 mg/day of the subject.

By 10 mg/day-25 mg/day it is meant that all hundredth, tenth and integer unit amounts within the range are specifically disclosed as part of the invention. Thus, 10.01, 10.02 . . . 24.99; 10.1, 10.2 . . . 24.9; and 11, 12 . . . 24 mg/day unit amounts are included as embodiments of this invention.

In another embodiment of the method, the amount of R(+)-6-(N-methyl,N-ethyl-carbamoyloxy)-N′-propargyl-1-aminoindan or a salt thereof administered is 0.14 mg/kg/day-0.42 mg/kg/day of the subject.

By 0.14 mg/kg/day-0.42 mg/kg/day it is meant that all hundredth and tenth unit amounts within the range are specifically disclosed as part of the invention. Thus, 0.15, 0.16, 0.17 . . . 0.41 mg/kg/day unit amounts are included as embodiments of this invention.

In another embodiment of the method, the amount of R(+)-6-(N-methyl,N-ethyl-carbamoyloxy)-N′-propargyl-1-aminoindan or a salt thereof administered is 0.15 mg/day of the subject.

In another embodiment of the method, the amount of R(+)-6-(N-methyl,N-ethyl-carbamoyloxy)-N′-propargyl-1-aminoindan or a salt thereof administered is 0.5 mg/kg of the subject, 0.784 mg/kg of the subject, 1 mg/kg of the subject, 1.5 mg/kg of the subject, 2 mg/kg of the subject, 3 mg/kg of the subject or 4 mg/kg of the subject.

In another embodiment, the method further comprises administering to the subject an antioxidant.

In another embodiment of the method, the neurodegenerative disease is Alzheimer's disease, dementia, mild cognitive impairment, Parkinson's disease, age-related macular degeneration, or amyotrophic lateral sclerosis.

In an embodiment of the method to treat dementia, the dementias include static dementia, senile dementia, presenile dementia, progressive dementia, vascular dementia or Lewy body dementia.

In an embodiment, the method slows the progression of an early stage neurodegenerative disease to a more advanced stage of the neurodegenerative disease in the subject.

In an embodiment, the method prevents the appearance of symptoms of a neurodegenerative disease in a subject predisposed to the disease.

The subject invention also provides a method of treating a subject afflicted with a neurodegenerative disease, comprising administering to the subject a less than cholinesterase-inhibitory amount or a less than monoamine oxidase-inhibitory amount of R(+)-6-(N-methyl,N-ethyl-carbamoyloxy)-N′-propargyl-1-aminoindan or a salt thereof effective to treat the subject.

In an embodiment, the method comprises administering to the subject a less than cholinesterase-inhibitory amount and less than monoamine oxidase-inhibitory amount of R(+)-6-(N-methyl,N-ethyl-carbamoyloxy)-N′-propargyl-1-aminoindan or a salt thereof effective to treat the subject.

In another embodiment, the method comprises administration of a salt of R(+)-6-(N-methyl,N-ethyl-carbamoyloxy)-N′-propargyl-1-aminoindan. The salt of R(+)-6-(N-methyl,N-ethyl-carbamoyloxy)-N′-propargyl-1-aminoindan may be the tartrate salt.

In a further embodiment of the method, the subject is a human.

In yet a further embodiment of the method with humans, the less than monoamine oxidase-inhibitory amount of R(+)-6-(N-methyl,N-ethyl-carbamoyloxy)-N′-propargyl-1-aminoindan is no more than 0.37 mg/kg/day of the subject, or a corresponding amount of the salt thereof.

In yet a further embodiment of the method with humans, the salt is the tartrate salt and the corresponding amount of the salt is no more than 0.46 mg/kg/day of the subject.

In yet a further embodiment of the method with humans, the less than cholinesterase-inhibitory amount of R(+)-6-(N-methyl,N-ethyl-carbamoyloxy)-N′-propargyl-1-aminoindan is no more than 2.3 mg/kg/day of the subject, or a corresponding amount of the salt thereof.

In yet a further embodiment of the method with humans, wherein the salt is the tartrate salt and the corresponding amount of the salt is no more than 2.9 mg/kg/day of the subject. Other amounts disclosed herein can also be used in this method.

In another embodiment of the method, the amount of R(+)-6-(N-methyl,N-ethyl-carbamoyloxy)-N′-propargyl-1-aminoindan or a salt thereof administered is 10 mg/day-25 mg/day of the subject.

By 10 mg/day-25 mg/day it is meant that all hundredth, tenth and integer unit amounts within the range are specifically disclosed as part of the invention. Thus, 10.01, 10.02 . . . 24.99; 10.1, 10.2 . . . 24.9; and 11, 12 . . . 24 mg/day unit amounts are included as embodiments of this invention.

In another embodiment of the method, the amount of R(+)-6-(N-methyl,N-ethyl-carbamoyloxy)-N′-propargyl-1-aminoindan or a salt thereof is 0.5 mg/kg of the subject, 0.784 mg/kg of the subject, 1 mg/kg of the subject, 1.5 mg/kg of the subject, 2 mg/kg of the subject, 3 mg/kg of the subject or 4 mg/kg of the subject.

In another embodiment of the method, the neurodegenerative disease is Alzheimer's disease, dementia, mild cognitive impairment, Parkinson's disease, age-related macular degeneration, or amyotrophic lateral sclerosis.

In another embodiment of the method, dementias include static dementia, senile dementia, presenile dementia, progressive dementia, vascular dementia or Lewy body dementia.

In another embodiment of the method, dementias include static dementia, senile dementia, presenile dementia, progressive dementia or vascular dementia.

The subject invention also provides a method of reducing oxidative stress in the brain of a subject afflicted with oxidative stress, comprising administering to the subject a less than cholinesterase-inhibitory amount or a less than monoamine oxidase-inhibitory amount of R(+)-6-(N-methyl,N-ethyl-carbamoyloxy)-N′-propargyl-1-aminoindan or a salt thereof effective to reduce oxidative stress in the brain of the subject.

In an embodiment, the method comprises administering to the subject a less than cholinesterase-inhibitory amount and a less than monoamine oxidase-inhibitory amount of R(+)-6-(N-methyl,N-ethyl-carbamoyloxy)-N′-propargyl-1-aminoindan or a salt thereof effective to treat the subject.

In another embodiment, the method comprises administration of a salt of R(+)-6-(N-methyl,N-ethyl-carbamoyloxy)-N′-propargyl-1-aminoindan.

In another embodiment of the method, the salt of R(+)-6-(N-methyl,N-ethyl-carbamoyloxy)-N′-propargyl-1-aminoindan is the tartrate salt.

In yet another embodiment of the method, the administration is acute administration.

In yet another embodiment of the method, subject is a human.

In yet another embodiment of the method with acute administration to humans, the less than monoamine oxidase-inhibitory amount of R(+)-6-(N-methyl,N-ethyl-carbamoyloxy)-N′-propargyl-1-aminoindan is no more than 1.7 mg/kg/day of the subject, or a corresponding amount of the salt thereof.

In yet another embodiment of the method with acute administration to humans, the salt is the tartrate salt and the corresponding amount of the salt is no more than 2.1 mg/kg/day of the subject.

In yet another embodiment of the method with acute administration to humans, the less than cholinesterase-inhibitory amount of R(+)-6-(N-methyl,N-ethyl-carbamoyloxy)-N′-propargyl-1-aminoindan is no more than 2.3 mg/kg/day of the subject, or a corresponding amount of the salt thereof.

In yet another embodiment of the method with acute administration to humans, the salt is the tartrate salt and the corresponding amount of the salt is no more than 2.9 mg/kg/day of the subject.

In a further embodiment of the method, the administration is chronic administration.

In a further embodiment of the method, the subject is a human.

In yet a further embodiment of the method with chronic administration to humans, the less than monoamine oxidase-inhibitory amount of R(+)-6-(N-methyl,N-ethyl-carbamoyloxy)-N′-propargyl-1-aminoindan is no more than 0.37 mg/kg/day of the subject, or a corresponding amount of the salt thereof.

In yet a further embodiment of the method with chronic administration to humans, the salt is the tartrate salt and the corresponding amount of the salt is no more than 0.46 mg/kg/day of the subject.

In yet a further embodiment of the method with chronic administration to humans, the less than cholinesterase-inhibitory amount of R(+)-6-(N-methyl,N-ethyl-carbamoyloxy)-N′-propargyl-1-aminoindan is no more than 2.3 mg/kg/day of the subject, or a corresponding amount of the salt thereof.

In yet a further embodiment of the method with chronic administration to humans, the salt is the tartrate salt and the corresponding amount of the salt is no more than 2.9 mg/kg/day of the subject.

In another embodiment of the method, the amount of R(+)-6-(N-methyl,N-ethyl-carbamoyloxy)-N′-propargyl-1-aminoindan or a salt thereof administered is 10 mg/day-25 mg/day of the subject.

By 10 mg/day-25 mg/day it is meant that all hundredth, tenth and integer unit amounts within the range are specifically disclosed as part of the invention. Thus, 10.01, 10.02 . . . 24.99; 10.1, 10.2 . . . 24.9; and 11, 12 . . . 24 mg/day unit amounts are included as embodiments of this invention.

In another embodiment of the method, the amount of R(+)-6-(N-methyl,N-ethyl-carbamoyloxy)-N′-propargyl-1-aminoindan or a salt thereof effective to treat the subject is 0.5 mg/kg of the subject, 0.784 mg/kg of the subject, 1 mg/kg of the subject, 1.5 mg/kg of the subject, 2 mg/kg of the subject, 3 mg/kg of the subject or 4 mg/kg of the subject.

In another embodiment of the method, the oxidative stress occurs in the hippocampus region of the brain.

In another embodiment of the method, the oxidative stress occurs in the astrocytes of the hippocampus region of the brain.

The subject invention also provides a method of treating a subject afflicted with mild cognitive impairment comprising administering to the subject an amount of R(+)-6-(N-methyl,N-ethyl-carbamoyloxy)-N′-propargyl-1-aminoindan or a salt thereof effective to treat the subject.

The subject invention also provides a method of treating a subject afflicted with mild cognitive impairment comprising administering to the subject a less than cholinesterase-inhibitory amount or a less than monoamine oxidase-inhibitory amount of R(+)-6-(N-methyl,N-ethyl-carbamoyloxy)-N′-propargyl-1-aminoindan or a salt thereof effective to treat the subject.

In an embodiment of the method, the less than cholinesterase-inhibitory amount or the less than monoamine oxidase-inhibitory amount of R(+)-6-(N-methyl,N-ethyl-carbamoyloxy)-N′-propargyl-1-aminoindan or the salt thereof is effective to delay the progress of MCI to Alzheimer's disease.

In another embodiment, the method comprises administering to the subject a less than cholinesterase-inhibitory amount and less than monoamine oxidase-inhibitory amount of R(+)-6-(N-methyl,N-ethyl-carbamoyloxy)-N′-propargyl-1-aminoindan or a salt thereof effective to treat the subject.

In another embodiment, the method comprises administration of a salt of R(+)-6-(N-methyl,N-ethyl-carbamoyloxy)-N′-propargyl-1-aminoindan.

In another embodiment of the method, the salt of R(+)-6-(N-methyl,N-ethyl-carbamoyloxy)-N′-propargyl-1-aminoindan is the tartrate salt.

In a further embodiment of the method, the subject is a human.

In yet a further embodiment of the method with humans, the amount of R(+)-6-(N-methyl,N-ethyl-carbamoyloxy)-N′-propargyl-1-aminoindan is no more than 0.37 mg/kg/day of the subject, or a corresponding amount of the salt thereof.

In yet a further embodiment of the method with humans, the salt is the tartrate salt and the corresponding amount of the salt is no more than 0.46 mg/kg/day of the subject.

In yet a further embodiment of the method with humans, the amount of R(+)-6-(N-methyl,N-ethyl-carbamoyloxy)-N′-propargyl-1-aminoindan is no more than 2.3 mg/kg/day of the subject, or a corresponding amount of the salt thereof.

In yet a further embodiment of the method with to humans, wherein the salt is the tartrate salt and the corresponding amount of the salt is no more than 2.9 mg/kg/day of the subject.

In another embodiment of the method, the amount of R(+)-6-(N-methyl,N-ethyl-carbamoyloxy)-N′-propargyl-1-aminoindan or a salt thereof administered is 10 mg/day-25 mg/day of the subject.

By 10 mg/day-25 mg/day it is meant that all hundredth, tenth and integer unit amounts within the range are specifically disclosed as part of the invention. Thus, 10.01, 10.02 . . . 24.99; 10.1, 10.2 . . . 24.9; and 11, 12 . . . 24 mg/day unit amounts are included as embodiments of this invention.

In another embodiment of the method, the amount of R(+)-6-(N-methyl,N-ethyl-carbamoyloxy)-N′-propargyl-1-aminoindan or a salt thereof is 0.5 mg/kg of the subject, 0.784 mg/kg of the subject, 1 mg/kg of the subject, 1.5 mg/kg of the subject, 2 mg/kg of the subject, 3 mg/kg of the subject or 4 mg/kg of the subject.

The subject invention also provides a pharmaceutical composition in unit dosage form comprising R(+)-6-(N-methyl,N-ethyl-carbamoyloxy)-N′-propargyl-1-aminoindan in an amount of up to 25 mg, or a corresponding amount of a pharmaceutically acceptable salt thereof, and a pharmaceutically acceptable carrier.

In an embodiment, the method comprises R(+)-6-(N-methyl,N-ethyl-carbamoyloxy)-N′-propargyl-1-aminoindan in an amount of 10 mg-25 mg or a corresponding amount of a pharmaceutically acceptable salt thereof, and a pharmaceutically acceptable carrier.

In a further embodiment of the method, the pharmaceutically acceptable salt is the tartrate salt.

This invention also provides a use of an amount of R(+)-6-(N-methyl,N-ethyl-carbamoyloxy)-N′-propargyl-1-aminoindan or a salt thereof in the manufacture of a medicament for preventing the appearance of symptoms of a neurodegenerative disease in a subject predisposed to the neurodegenerative disease or for slowing the progression of an early stage neurodegenerative disease to a more advanced stage of the neurodegenerative disease. The amount of R(+)-6-(N-methyl,N-ethyl-carbamoyloxy)-N′-propargyl-1-aminoindan or salt thereof in the medicament is effective to prevent the appearance of symptoms of the disease or to slow the progression of the disease. The amount of R(+)-6-(N-methyl,N-ethyl-carbamoyloxy)-N′-propargyl-1-aminoindan or salt thereof may also be a less than cholinesterase-inhibitory amount or a less than monoamine oxidase-inhibitory amount. Other embodiments disclosed herein may also be implemented in the context of this use.

This invention further provides a use of an amount of R(+)-6-(N-methyl,N-ethyl-carbamoyloxy)-N′-propargyl-1-aminoindan or a salt thereof in the manufacture of a medicament for reducing oxidative stress in the brain of a subject afflicted with such oxidative stress. Other embodiments disclosed herein including the amounts of R(+)-6-(N-methyl,N-ethyl-carbamoyloxy)-N′-propargyl-1-aminoindan or salt thereof in the medicament may be readily implemented in the context of this use.

This invention additionally provides a use of an amount of R(+)-6-(N-methyl,N-ethyl-carbamoyloxy)-N′-propargyl-1-aminoindan or a salt thereof for manufacturing a medicament for treating mild cognitive impairment in a subject afflicted therewith. Other embodiments disclosed herein including the amount of R(+)-6-(N-methyl,N-ethyl-carbamoyloxy)-N′-propargyl-1-aminoindan or salt thereof in the medicament may be readily implemented in the context of this use.

The compound R(+)-6-(N-methyl,N-ethyl-carbamoyloxy)-N′-propargyl-1-aminoindan may be prepared as pharmaceutical compositions particularly useful for the prevention or treatment of neurodegenerative diseases or of oxidative stress. For example, the compositions can also contain adjunct therapy agents for the neurodegenerative disease or additional antioxidants.

Such compositions may comprise ladostigil or pharmaceutically acceptable salts thereof, together with pharmaceutically acceptable carriers and/or excipients. Dosages of unit dosage forms may be in the range of, e.g., 1-25 mg, or preferably 5-20 mg. In the practice of this invention, pharmaceutically acceptable salts include, but are not limited to, the mesylate, maleate, fumarate, tartrate, hydrochloride, hydrobromide, esylate, p-tolunesulfonate, benzoate, acetate, phosphate and sulfate salts.

The compositions may be prepared as medicaments to be administered orally, parenterally, rectally or transdermally. Suitable forms for oral administration include tablets, compressed or coated pills, dragees, sachets, hard or soft gelatin capsules, sublingual tablets, syrups and suspensions; for parenteral administration the invention provides ampoules or vials that include an aqueous or non-aqueous solution or emulsion; for rectal administration there are provided suppositories with hydrophilic or hydrophobic vehicles; and for topical application as ointments and transdermal delivery there are provided suitable delivery systems as known in the art.

Specific examples of pharmaceutical acceptable carriers and excipients that may be used to formulate oral dosage forms of the present invention are described, e.g., in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators include, without limitation, starch, methyl cellulose, agar, bentonite, xanthan gum, croscarmellose sodium, sodium starch glycolate and the like.

As used herein, a “pharmaceutically acceptable” carrier is one that is suitable for administration to humans and/or animals without undue adverse side effects (such as toxicity, irritation, and allergic response) commensurate with a reasonable benefit/risk ratio.

As used herein, a “neurodegenerative disease” refers to a disease in which degeneration occurs in either gray or white matter, or both, of the nervous system. Thus, such a disease can be diabetic neuropathy, senile dementias, Alzheimer's disease, Mild Cognitive Impairment (MCI), Parkinson's Disease, facial nerve (Bell's) palsy, glaucoma, Huntington's chorea, amyotrophic lateral sclerosis (ALS), status epilepticus, non-arteritic optic neuropathy, intervertebral disc herniation, vitamin deficiency, prion diseases such as Creutzfeldt-Jakob disease, carpal tunnel syndrome, peripheral neuropathies associated with various diseases, including but not limited to, uremia, porphyria, hypoglycemia, Sjorgren Larsson syndrome, acute sensory neuropathy, chronic ataxic neuropathy, biliary cirrhosis, primary amyloidosis, obstructive lung diseases, acromegaly, malabsorption syndromes, polycythemia vera, IgA and IgG gammapathies, complications of various drugs (e.g., metronidazole) and toxins (e.g., alcohol or organophosphates), Charcot-Marie-Tooth disease, ataxia telangectasia, Friedreich's ataxia, amyloid polyneuropathies, adrenomyeloneuropathy, Giant axonal neuropathy, Refsum's disease, Fabry's disease and lipoproteinemia.

As used herein, “predisposed” to a neurodegenerative disease or to oxidative stress in the brain may refer to a genetic, familial or chemically-induced predisposition.

As used herein, a “less than monoamine-oxidase inhibitory amount” refers to an amount that causes no more than 25% MAO inhibition upon acute administration to a subject, or no more than 30% MAO inhibition during chronic administration to a subject.

A “less than cholinesterase-inhibitory amount” refers to an amount that causes no more than 30% ChE inhibition upon acute administration to a subject, or no more than 30% ChE inhibition during chronic administration to a subject.

In the methods described, the administration may be periodic or regular. Thus, the administration may be in a single unit dose of the desired amount per a given period, e.g. once per day, week, month, etc, or it may be composed of multiple dosages adding up to the desired amount over the period. For example, administration may be once, twice, three or four times every day, or every 5 days, every week, 2 weeks, 3 weeks, month, 2 months, 3 months, 6 months or year.

As used herein, “acute” administration refers to a single administration to a subject.

As used herein, “chronic” administration refers to more than one administration to a subject, each administration occurring before the previous is completely cleared from the subject. In an embodiment, chronic administration may be administration seven consecutive times at regular intervals, e.g., daily.

As used herein, a subject “afflicted” with neurodegenerative disease or with oxidative stress in the brain means the subject has been diagnosed with neurodegenerative disease or oxidative stress in the brain.

Thus, this invention provides a method for delaying or preventing development of, or appearance of symptoms of, a neurodegenerative disease in a subject predisposed to such disease, as well as a method for slowing or suppressing progression of such a disease in a subject afflicted with an early stage of such disease.

### EXPERIMENTAL DETAILS

Monoamine Oxidase (MAO) Inhibition Assay

The level of MAO (MAO-A and MAO-B) inhibition can be determined by routine methods, including those detailed below.

In vivo

Rats and mice are treated by subcutaneous (s.c.) or intraperitoneal (i.p.) injection or oral (p.o.) administration of the compound to be tested. Animals are decapitated approximately 2 hours after drug treatment and MAO activity is determined in the brain, intestine, and/or liver. MAO activity is expressed as percentage of enzyme activity relative to a control group administered with vehicle only. Effective doses producing 50% MAO inhibition (ED₅₀ values) are calculated from inhibition curves.

Repeated treatment by ladostigil (e.g. daily doses for 7 days) can be used to calculate inhibition achieved through chronic administration.

In the case of ladostigil tartrate, this method was used to determine that amounts up to 20.4 mg/kg of ladostigil base (acute administration) and up to 4.5 mg/kg of ladostigil base (chronic administration) cause no more than 25% MAO inhibition (both MAO-A and MAO-B).

In vitro

The MAO enzyme obtained from a homogenate of rat brain in 0.3M sucrose, which was centrifuged at 600 g for 15 minutes. The supernatant is diluted appropriately in 0.05M phosphate buffer, and pre-incubated with serial dilutions of test compounds for 20 minutes at 37° C. ¹⁴C-Labeled substrates (2-phenylethylamine, hereinafter PEA; 5-hydroxytryptamine, hereinafter 5-HT) are then added, and the incubation continued for a further 20 minutes (PEA), or 30-45 minutes (5-HT). Substrate concentrations used are 50 μM (PEA) and 1 mM (5-HT). In the case of PEA, enzyme concentration is chosen so that not more than 10% of the substrate is metabolized during the course of the reaction. Deaminated products are extracted into toluene-ethyl acetate (1:1 v/v) containing 0.6% (w/v) 2,5-diphenyloxazole (ppo) prior to determination by liquid scintillation counting. Radioactivity in the eluate indicates the production of neutral and acidic metabolites formed as a result of MAO activity. Activity of MAO in the sample is expressed as a percentage of control activity in the absence of inhibitors after subtraction of appropriate blank values. The activity determined using PEA as substrate is referred to as MAO-B, and that determined using 5-HT as MAO-A. Concentrations of inhibitor producing 50% inhibition of substrate metabolism (IC₅₀) are calculated from the inhibition curves.

Acetylcholinesterase (ChE) Inhibition Assay

The level of ChE inhibition can be determined by routine methods, including those detailed below.

In vivo

In vivo measurement of ChE inhibition is similar to that of in vivo measurement of MAO inhibition, except that cynomolgus monkeys are treated, and tissues are examined for brain, plasma and erythrocytes ChE inhibition.

In the case of ladostigil tartrate, this method was used to determine that amounts up to 7.0 mg/kg ladostigil base (acute or chronic administration) cause no more than 30% inhibition.

In vitro

Human erythrocyte acetylcholinesterase (type XIII, Sigma Israel), is prepared in a stock solution of 1 U/ml, containing Triton (1%) and bovine serum albumin (0.05%) in phosphate buffer (pH 8). The enzyme (0.05U) is incubated with 3-5 different concentrations of test compound (in triplicate) for periods of from 15 to 60 minutes at 37° C. The substrate acetylthiocholine (0.075M) and 5,5′-dithiobis-(2-nitrobenzoic acid) (DTNB, 0.01M) are then added and the rate of hydrolysis of the substrate which yields a yellow product monitored spectrophotomerically at 412 nM (Ellman et al., Biochem Pharmacol. (1961) 7: 88-95). The percentage inhibition of ChE by each concentration of drug is calculated by comparison with that of enzyme in the absence of drug. The concentration of each drug that inhibits ChE by 50% (IC₅₀) at the time of peak activity is then calculated.

A standard method for converting a dosage used in animals to a dosage appropriate for human use is publicly available (Guidance for Industry: Estimating the Maximum Safe Starting Dose in Initial Clinical Trials for Therapeutics in Adult Healthy Volunteers, U.S. Dept. HHS/FDA/CDER (July 2005), at http://www.fda.gov/cder/guidance/5541fnl.doc). The dose conversion is species dependent. Interspecies dose conversion consists of dividing the animal dosage by a standard factor in order to derive the dosage for human use. The recommended standard factor for converting to the human equivalent dose for an average 60 kg human based on the animal species is, e.g., 12.3 for mice, 6.2 for rats, 3.1 for cynomolgous monkeys. Alternatively, the human equivalent dose (HED) can be obtained using the following formula: HED=animal dose in mg/kg * (animal weight in kg/human weight in kg)ˆ0.33.

Animal Testing

Streptozotocin (“STZ”) is known to cause oxidative stress in pancreatic beta cells when injected parenterally (Takasu et al., Streptozotocin- and alloxan-induced H₂O₂ generation and DNA fragmentation in pancreatic islets, Diabetes (1991) 40:1141-5). Previous studies by the inventors have shown that a single bilateral icv injection of STZ causes impairment of episodic memory as measured by the object recognition test 2 weeks later, which is still present after 8 weeks. Memory deficits are seen 3-8 weeks later, particularly after 3 icv injections of STZ (Lannert and Hoyer, 1998) and can be reduced by chronic treatment with antioxidants, melatonin and resveratrol (Sharma and Gupta, 2001, 2002), supporting a role of ROS in their aetiology.

Previous studies by the inventors have also shown that STZ induces oxidative nitrative stress in the stratum oriens of the hippocampus after one week, and that a single STZ injection reduces the levels of low molecular weight antioxidants in the fornix and corpus callosum. In a detailed histological examination of the brain after intracerebroventricular (icv) injection of streptozotocin (STZ) in rats it was found that microglial activation occurs in discrete areas (Shoham et al., 2003). Icv STZ causes activation of astrocytes and microglia in the hippocampus and at the site of STZ injection, which leads to the production of nitric oxide (NO) and ultimately reactive oxygen species (O₂— and OH—) (Simmons et al., Induction of nitric oxide synthase in glial cells, J. Neurochem. (1992) 59:897-905).

Activated astrocytes express excitatory amino acid receptors, take up glutamate, and are also at risk of oxidative stress (Ouyang and Giffard, 2004). Thus, a common origin of oxidative stress in hippocampus could be excess excitatory stimulation (glutamate-GABA imbalance) that involves intracellular calcium dysregulation that can lead to disruption of intracellular anti-oxidant defense systems (Lipton et al., Excitatory amino acids as a final common pathway for neurological disorders, N. Engl. J. Med. (1994) 330:613-22).

Activation followed by a loss of astroglia can result in dysregulation of glia to neuron signaling leading (Kielian et al., Effects of neuroinflammation on glia-glia gap junctional intercellular communication: a perspective, Neurochem. Int. (2004) 45:429-36). This may induce a memory deficit by slowing impulse conduction in myelinated neurons in the cortex and hippocampus and eventually lead to a reduction in hippocampal cholinergic transmission. Thus, STZ-induced oxidative stress was selected as a model to test the anti-oxidant potential of ladostigil.

### EXAMPLE 1

The study was performed on male Sprague-Dawley rats (Harlan, Jerusalem) weighing 320-340 g, aged 4 months, according to the guidelines of the University Committee for Institutional Animal Care and based on those of the National Institutes of Health, USA. The rats were housed for one week prior to surgery in the Animal House at an ambient temperature of 21±1° C. and a 12 hr diurnal light cycle (lights on at 0700 hr).

Male Sprague-Dawley rats (30) weighing 320-340 g, were anaesthetized by an intraperitoneal (i.p.) injection of Equithesin 0.3 ml/kg. STZ (0.5 mg) in 2 μl artificial cerebrospinal fluid (CSF) was injected into each lateral ventricle of 20 rats. The stereotaxic co-ordinates for icv injection were: 0.9 mm posterior, 1.8 mm lateral and 3.8 mm ventral from the bregma level. Control rats (10) received a bilateral injection of 2 μl artificial CSF. Half of the STZ-injected rats were given ladostigil (1 mg/kg) orally in a volume of (1 ml/kg) one week before, and for one week after after icv STZ. The remainder, received 1 ml/kg water. The rats were anaesthetized with sodium pentobarbitone, perfused transcardially and the brains processed for immunocytochemistry as described below.

Histopathology

The brains were sectioned in a cryostat in 30 μm thick slices and stained immunochemically with the following markers: Nitrotyrosine, a marker of nitrative/oxidative stress, was stained with rabbit anti-nitrotyrosine from Upstate (Lake Placid, N.Y., USA), diluted 1:100. Glial fibrillary acidic protein (GFAP), a marker of astrocyte activation, was stained with mouse antibody, clone GA-5 from Sigma (Rehovot, Israel), diluted 1:500. 1:250. For rabbit antibodies, the secondary donkey anti-rabbit conjugated with horseradish peroxidase from Chemicon (Temecula, Calif., USA), was diluted 1:200. For mouse anti-GFAP, the secondary goat anti mouse conjugated with horseradish peroxidase from Sigma (Rehovot, Israel), was diluted 1:100. Colour development was obtained by reaction with diaminobenzidine and hydrogen peroxide as described in Shoham et al., Intracerebroventricular injection of streptozotocin causes neurotoxicity to myelin that contributes to spatial memory deficits in rats, Exp. Neurol. (2003) 184:1043-52. Images were obtained by bright field microscopy and quantitative assessments of appropriate markers were made by means of densitometric measurements with AnalySIS software.

Confocal Microscopy for Correlation Between Astrocyte Activation and Oxidative Stress

Brain sections were incubated with both rabbit anti NT and mouse anti GFAP. Instead of the DAB-peroxidase color reaction, the secondary antibodies were conjugated with fluorescein isothiocyanate (FITC) (NT) or with rhodamine isothiocyanate (RITC) (GFAP).

Quantification with Image Analysis

NT-immunoreactive astrocytes were seen in the stratum oriens, stratum, radiatum and alveus of the hippocampus. Rectangular fields measuring 200 μm×100 μm were sampled from these areas in two sections containing the hippocampus from each rat. The percentage area in which nitrotyrosine immunoreactivity was seen was computed and averaged between the two sections.

Statistical Analysis

Densitometic data were analyzed by univariate ANOVA followed by Duncan's post hoc test.

TABLE 1*

| | | Nitrotyrosine Immunoreactivity | | |
|---|---|---|---|---|
| | | | Stratum | Stratum |
| Rat | Treatment | Alveus | oriens | radiatum |
| Cl | CSF | 0 | 0 | 0 |
| C2 | n = 6 | 14.5 | 5.5 | 4 |
| C3 | | 2.5 | 5 | 5 |
| C4 | | 12 | 7 | 1.5 |
| C5 | | 6.5 | 11.5 | 0.5 |
| C6 | | 0 | 1 | 0.5 |
| | Mean ± SEM | 5.9 ± 2.5 | 5.0 ± 1.7 | 1.9 ± 0.8 |
| S1 | STZ | 30 | 40 | 46 |
| S2 | n = 7 | 33.5 | 60 | 48 |
| S3 | | 22 | 33 | 52 |
| S4 | | 23.5 | 55.5 | 69.5 |
| S5 | | 20 | 43 | 40.5 |
| S6 | | 20 | 28 | 25.5 |
| S7 | | 38.5 | 50.5 | 53 |
| | Mean ± SEM | 26.8 ± 2.8 | 44.3 ± 4.4 | 47.8 ± 5.0 |
| SL1 | STZ + | 4 | 14.5 | 1.5 |
| SL2 | ladostigil | 9.5 | 19 | 22 |
| SL3 | n = 5 | 7 | 5.5 | 3 |
| SL4 | | 9 | 8.5 | 7.5 |
| SL5 | | 15 | 20.5 | 11.5 |
| | Mean ± SEM | 8.9 ± 1.8 | 13.6 ± 2.9 | 9.1 ± 3.7 |

*Data represent mean of two sections per rat

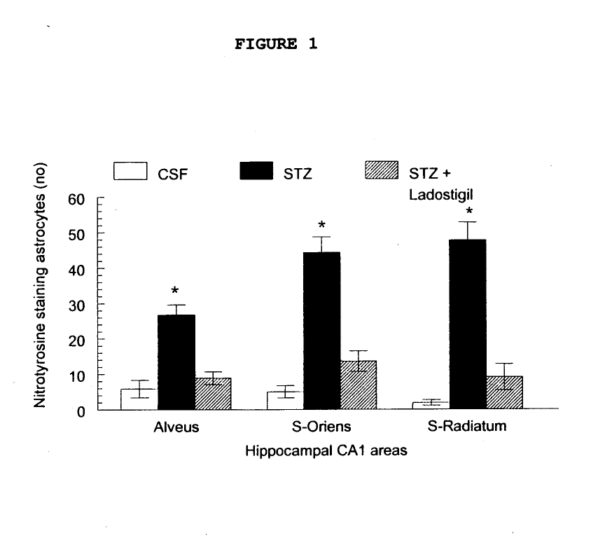

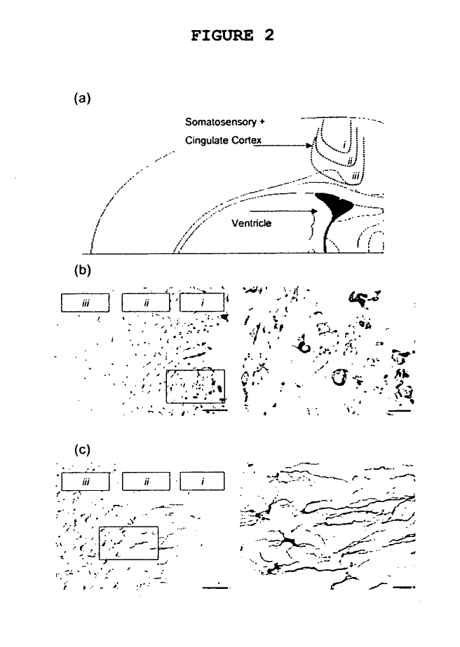

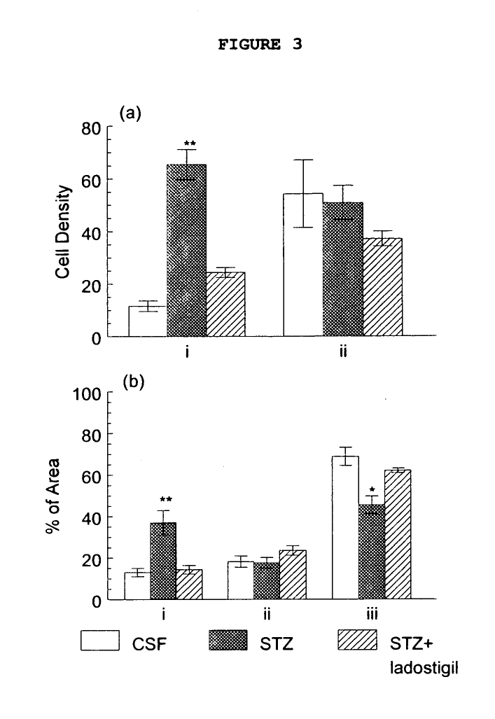

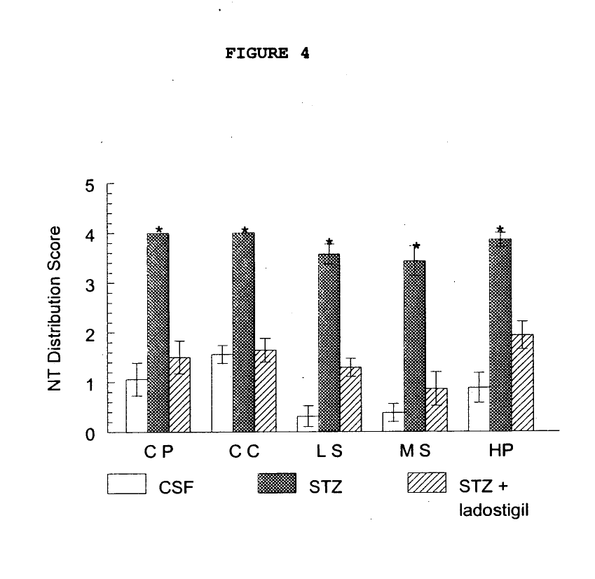

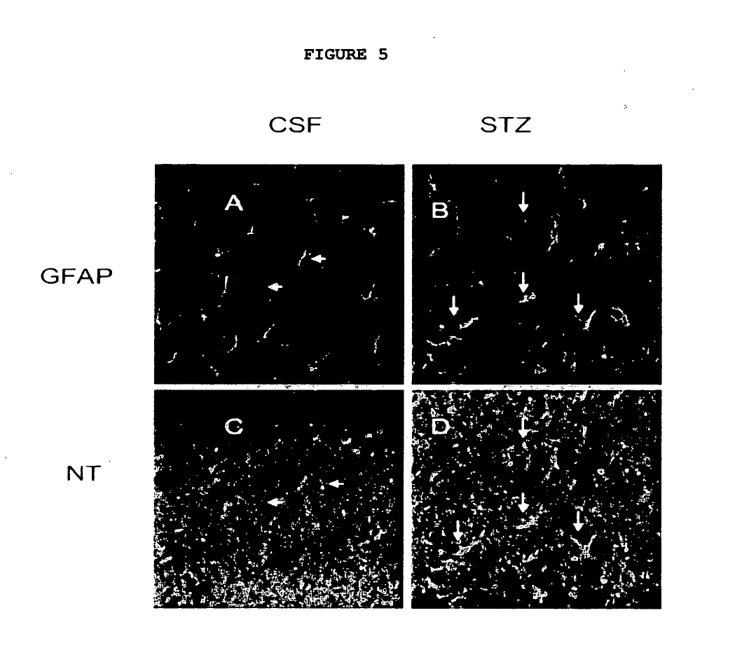

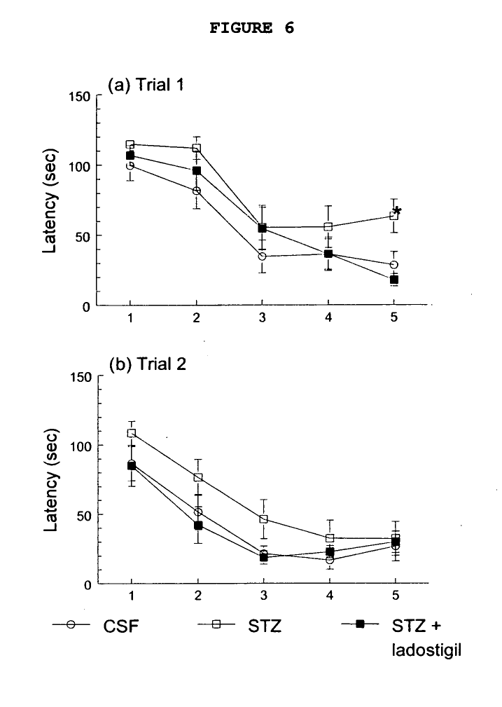

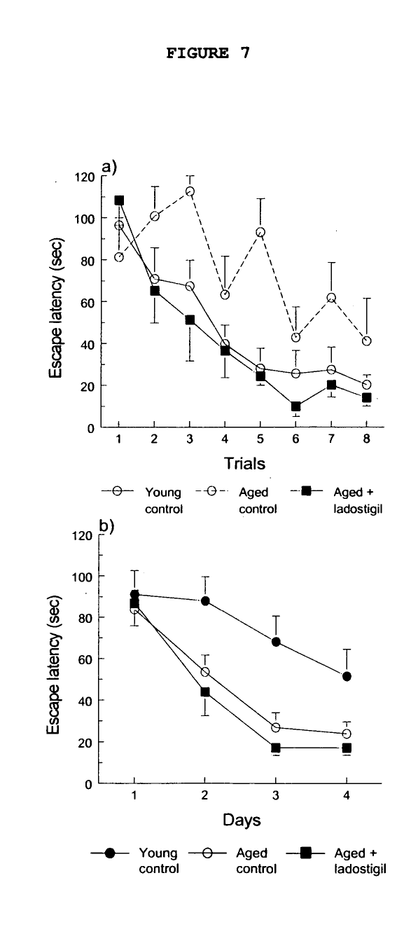

One week after a single icv injection of STZ a significant increase in nitrotyrosine staining was seen in the stratum oriens, alveus and stratum radiatum of the hippocampal CA1 field. Confocal microscopy revealed that the nitrotyrosine immunoreactivity occurred in reactive astrocytes. Ladostigil completely prevented the increase in nitrotyrosine immunoreactivity in astrocytes in three areas of the CA1 region of the hippocampus (FIG. 1).

Discussion

A single bilateral icv injection of streptozotocin (“STZ”) induces oxidative nitrative stress in the stratum oriens of the hippocampus after one week. The present invention extends this finding to two adjacent areas in the CA1 region of the hippocampus, the alveus and stratum. The induction of nitrotyrosine immunoreactivity suggests a unique vulnerability of this region to some STZ-induced processes that involve oxidative stress. Specifically, STZ causes activation followed by a loss of astrocytes in this brain region, thereby increasing the likelihood of glutamate overactivity.

Daily oral administration of ladostigil (1.0 mg/kg), from one week before, to one week after icv STZ injection prevented the increase in nitrotyrosine immunoreactivity in astrocytes in 3 hippocampal fields. This suggests that a low dose of ladostigil can reduce oxidative-nitrative stress in astrocytes in a defined hippocampal region, which may lessen the reduction in signaling in hippocampal pyramidal cells by STZ thereby reducing the memory deficit.

### EXAMPLE 2

The study was performed on male Sprague-Dawley rats (Harlan, Jerusalem) weighing 320-340 g, aged 4 months, according to the guidelines of the University Committee for Institutional Animal Care and based on those of the National Institutes of Health, USA. The rats were housed for one week prior to surgery in the Animal House at an ambient temperature of 21±1° C. and a 12 hr diurnal light cycle (lights on at 0700 hr).

Male Sprague-Dawley rats (63) weighing 320-340 g, aged 4 months, were anesthetized by an intraperitoneal injection of Equithesin 0.3 ml/kg. STZ (0.5 mg) dissolved in 2 μl artificial cerebrospinal fluid (CSF) was injected into each lateral ventricle of 45 rats. The stereotaxic coordinates for icv injection were: 0.9 mm posterior, 1.8 mm lateral and 3.8 mm ventral from the bregma level. Control rats (18) received a bilateral injection of 2 μl artificial CSF. STZ-injected rats (25) were given ladostigil (1 mg/kg of the hemitartrate salt/day) orally in a volume of (1 ml/kg) from one week before, until 1 or 8 weeks after icv STZ. The remaining 20 rats received 1 ml/kg water. The rats were housed two per cage with free access to food and water until the behavioral, enzymatic and cytochemical measurements were completed.

Immunohistochemistry

One week after icv injection, 8 rats injected with icv CSF, 7 rats given STZ alone, and 7 treated with ladostigil were anesthetized with sodium pentobarbitone, perfused transcardially and the brains processed for immunocytochemistry. This procedure was repeated in another 6 rats of each group, 8 weeks after icv injection, when the rats had been tested for memory deficits as described below. Floating sections 30 μm thick were prepared from the brains in a cryostat and were cryopreserved as described in Shoham et al., supra.

Glial Activation and Nitrative Stress

Nitrotyrosine, (NT) a marker of nitrative/oxidative stress, was stained with rabbit anti-nitrotyrosine from Upstate (Lake Placid, N.Y., USA), diluted 1:100 followed by the secondary donkey anti-rabbit conjugated with horseradish peroxidase from Chemicon (Temecula, Calif., USA), was diluted 1:200. GFAP, a marker of astrocyte activation, was stained first with mouse antibody, clone GA-5 from Sigma (Rehovot, Israel), diluted 1:500 then with the secondary goat anti-mouse conjugated with horseradish peroxidase from Sigma (Rehovot, Israel), diluted 1:100. Microglia were stained by incubation with biotinylated lectin (Griffonia Bandeirea Simplicifolia, Vector laboratories, Burlingame Calif.), followed by extravidin conjugation with horseradish peroxidase. Color was developed by reaction with diaminobenzidine and hydrogen peroxide as previously described in Shoham et al., supra. Images were obtained by bright field microscopy and quantitative assessments of appropriate markers were made using AnalySIS software.

Cholinergic Markers

Cholinergic neurons were stained for choline acetyl transferase (ChAT) with goat anti ChAT 1:200, (Chemicon, Temecula, Calif., USA), vesicular acetylcholine transporter (VAChT) with rabbit anti VAChT (Sigma, Rehovot, Israel) 1:100, and for nerve growth factor receptor (NGFR) rabbit anti NGFR (Alomone labs, Jerusalem, Israel) 1:100. The secondary antibody solutions were donkey anti goat and donkey anti rabbit 1:200, each conjugated with horseradish peroxidase (Chemicon, Temecula, Calif., USA). The final color reaction was developed as previously described in Shoham et al., supra.

Confocal Microscopy

To detect evidence of oxidative-nitrative stress in activated astrocytes, brain sections were incubated with both rabbit anti NT and mouse anti GFAP. Instead of the DAB-peroxidase color reaction, the secondary antibodies were conjugated with fluorescein isothiocyanate for NT or with rhodamine isothiocyanate for GFAP. To explore the influence of STZ injection on microglial and astrocytes, brain sections were incubated with both mouse anti-GFAP (for astrocytes) and biotin-labeled isolectin B4 (for microglia). The secondary antibodies were fluorescein isothiocyanate for GFAP and streptavidin Cy3 for isolectinB4.

Quantification with Image Analysis

Three distinct zones were distinguished in the vicinity of the cortical injection site after icv STZ injection containing astrocytes and microglia with differing morphology. Quantification of microglia and astrocytes in the three zones was made in sections sampled in a range of P0.2-0.8 mm from the bregma. The area of each zone containing the different types of astrocytes was measured and the percentage that each zone occupied in the wound area was calculated (Statistica software, StatSoft, USA). The classes of microglia were counted separately in each zone. The number of cells was divided by the zone area to obtain a measure of cell density.

For each quantitative analysis of NT-immunoreactive astrocytes, 3 sections were sampled from each rat. In 4 brain regions bordering the lateral ventricles and in the CA1 region of the hippocampus NT immunoreactivity was assessed according the following scoring system: 0=No NT-positive astrocytes; 1=NT-positive staining in astrocytic fibers but not in cell bodies; about 7 astrocytes per 40× field; 2=NT-positive staining in astrocytic fibers and in cell bodies; about 20 astrocytes per 40× field; 3=NT-positive astrocytes are present throughout the region; 4=NT-positive astrocytes are present throughout the region and intensely stained.

Cholinesterase and Monoamine Oxidase Activity

The effect of chronic ladostigil treatment on ChE activity in the cortex and hippocampus of rats was determined one week after icv STZ in 12 rats (3 treated with water and 9 with ladostigil). The effect of acute treatment with ladostigil 1 or 17 mg/kg was determined 2 hrs after drug administration. The rats were sacrificed by cervical dislocation, 120 min after the last dose of drug as maximal ChE inhibition was found at this time (Weinstock et al., 2000). The brain was rapidly removed, washed with saline on ice and the cortex, hippocampus and striatum dissected out, quickly weighed and stored at −70° C. until assay. Total ChE activity in the cortex and hippocampus was measured as described in Wang et al., Gender differences in the effect of rivastigmine on brain cholinesterase activity and cognitive function in rats, Neuropharmacology (2000) 39:497-506, using the spectrophotometric method of Ellman et al., A new and rapid calorimetric determination of acetylcholinesterase activity, Biochem. Pharmacol. (1961) 7:88-95 with acetylthiocholine (Sigma Ltd) as a substrate using homogenate of 100 mg tissue in 1 ml phosphate buffer. To determine only butyrylcholinesterase (BuChE) activity the amounts of tissue/ml of buffer were increased 3-fold and butyrylthiocholine (Sigma Ltd) was used as a substrate. MAO A and B activity was measured in homogenates of striatum as described in Weinstock et al., Limited potentiation of blood pressure response to oral tyramine by brain-selective monoamine oxidase A-B inhibitor, TV-3326 in conscious rabbits, Neuropharmacology (2002) 43:999-1005.

Object and Place Recognition

Episodic memory in the object recognition test was still intact 2 but not 4 weeks after a single icv STZ injection (Table 3). Therefore, rats that were treated with ladostigil or water were tested for object and place recognition 4 weeks after icv injection of STZ or CSF. The object recognition test measured non-spatial working memory with characteristics of episodic memory that is primarily affected in senile dementia (Bartolini et al., Aniracetam restores object recognition impaired by age, scopolamine, and nucleus basalis lesions, Pharmacol. Biochem. Behav. (1996) 53:277-83). The test depended on intact cortical and hippocampal function (Winters et al., Double dissociation between the effects of peri-postrhinal cortex and hippocampal lesions on tests of object recognition and spatial memory: heterogeneity of function within the temporal lobe, J. Neuroscience (2004) 24:5901-8), while place recognition, another manifestation of spatial memory, was dependent on intact hippocampal function. The experiments were carried out as previously described in Mumby et al., Hippocampal damage and exploratory preferences in rats: memory for objects, places, and contexts, Learning Memory (2002) 9:49-57, in a box made of dark Perspex (60×60 cm and 50 cm high), covered with a dark Perspex lid. In order to increase their interest in the objects, rats were familiarized with the test arena by placing them in it for 5 min on each of 3 successive days and the object recognition test performed on the 4^{th} day. Two identical objects were placed in the arena and the time taken by the rats to explore each object during a period of 3 min was recorded. One hour later, the objects were replaced by one that was identical to the two used in the first test and a second one that was different from them and the time taken to explore each of them was again recorded. The following day, the place recognition test was carried out with two identical objects as described above but different from those used previously. One hour later, the one of the two objects was moved to another position in the arena as described in Mumby et al., supra.

Spatial Memory in Water Maze

Two weeks after the place recognition task the rats were tested for spatial memory in the Morris water maze (MWM), which consisted of a circular pool (150 cm in diameter, 60 cm high) filled to depth of 30 cm with water at a temperature of 22±1° C. A transparent glass escape platform (20 cm in diameter) was placed 1 cm below the surface, midway between the centre and rim of the pool in one quadrant where it remained for all the acquisition trials. For data analysis the tank was divided into four quadrants, north (N), south (S), east (E) and west (W). Both collection and analysis of the data were performed using an automated video-tracking system (HVS, Ltd). To begin each trial the rat was placed into the water, facing the maze wall, from one of four start positions evenly spaced around the pool (N, S, E & W). Start positions were chosen randomly at the beginning of each day for all rats. If the rat failed to find the escape platform within 120 s it was placed on it for 10 sec and then removed from the pool. The rat was given two trials a day for 5 days between 10:00 and 14:00 hr with an inter-trial interval of 15 min.

Data Analysis

Group means of 3 measures in the object and place recognition tests, duration of exploration in first and second phases and discrimination index were submitted to one way ANOVA. Mean daily escape latencies for trial 1 and trial 2 in the MWM in each group were analyzed by repeated measures ANOVA for DAY and GROUP followed by Duncan's post hoc test where appropriate. Histological data were analyzed by univariate ANOVA followed by Duncan's post hoc test. All values represent means±SEM.

Results

Changes in microglia and astrocytes in vicinity of cannula penetration site.

The three zones of gliosis that could be distinguished in the motor and cingulate cortex close to the cannula penetration site seven days after icv STZ are shown in FIG. 2. Zone i) contained microglia with round shaped soma devoid of processes that resembled macrophages but was almost free of astrocytes; zone ii) had activated microglia with polymorphic shaped soma and a variety of fibrous processes and activated astrocytes with elongated processes but no soma; zone iii) had resident microglia resembling those in the CSF-injected controls and reactive astrocytes with thickened processes and filled soma. Ladostigil prevented the changes both in microglia and in astrocytes in zones i) and iii) that resulted from icv injection of STZ but had no effect on the density of fibrous microglia in zone ii) which was similar to that in CSF-injected controls (FIGS. 3a, b).

In the hippocampus, the effect of STZ on glial cells was less well defined. Reactive astrocytes were present in scattered groups most of which were found the CA1 region. They were also seen in the corpus callosum, medial and lateral septum close to the lateral ventricle, while other areas were almost devoid of astrocytes. In all these areas the astrocytes showed a significant increase in NT immunoreactivity (FIGS. 4 and 5). Ladostigil treatment reduced both astrocyte activation and NT immunoreactivity in these brain regions to the level of that seen in CSF-injected controls (FIGS. 4 and 6).

Eight weeks after icv STZ injection, the cannula penetration site contained scar tissue that was filled with tightly packed astrocytic fibers, including the zone previously devoid of them, that were not reactive. Around the scar there were a few reactive astrocytes but no reactive microglia, rounded or fibrous, in or around the scar. No differences were detected in scar morphology in rats injected with icv STZ with or without ladostigil. There were also no signs of NT immunoreactivity in astrocytes in any of the brain regions in which this was present earlier.

Cholinergic Markers

No change in the number or morphology of cholinergic neurons was detected in any of the basal forebrain nuclei, medial septum, diagonal band and nucleus basalis magnocellularis one or eight weeks after icv STZ injection. There was also no change in the density of cholinergic terminals in the hippocampus based on VAChT immuno-reactive varicosities and axonal processes.

Cholinesterase and Monoamine Oxidase Activity

The effect of chronic treatment with ladostigil (1 mg/kg) on total enzyme activity and that of BuChE is shown in Table 2.

TABLE 2
Inhibition of cholinesterase and monoamine
oxidase by ladostigil

| Brain | Time | | | | MAO-B |
|---|---|---|---|---|---|
| region | (min) | ChE (%) | BuChE (%) | MAO-A (%) | (%) |
| Cortex | 60 | 16.1 ± 4.7* | 16.2 ± 2.5* | NT | NT |
| | 120 | 23.6 ± 3.1* | 15.7 ± 3.2* | NT | NT |
| Hippo- | 60 | 4.9 ± 2.6 | 7.3 ± 2.5 | NT | NT |
| campus | 120 | 5.9 ± 3.2 | 5.4 ± 2.8 | NT | NT |
| Stri- | 60 | NT | NT | 2.8 ± 2.5 | 11.3 ± 1.4* |
| atum | 120 | NT | NT | 3.8 ± 2.0 | 15.1 ± 3.4* |
| Plasma | 60 | NT | NT | NT | NT |
| | 120 | 20 ± 2% | NT | NT | NT |

NT = not tested.
Significantly different from untreated rats, *P < 0.05.

A relatively low but significant degree of inhibition was found in the cortex; (24%) of total enzyme activity and 16% of BuChE, seven days after icv STZ and 1 or 2 hrs after the last dose of ladostigil. ChE activity in the plasma was inhibited by 20% However, no significant inhibition of either enzyme was detected in the hippocampus. Ladostigil treatment also had no effect on striatal MOA-A activity and only reduced that of MAO-B by 15% at 2 hrs (Table 2).

Acute administration of ladostigil (1 mg/kg) only inhibited ChE in the cortex by 7.8±1.5% but not in the hippocampus or plasma, while 17 mg/kg inhibited ChE in both brain regions and in plasma by 40-42%.

Object and Place Recognition

The results of the object and place recognition tests are shown in Table 3.

TABLE 3
Discrimination indices from object^{§} and place
recognition^{‡} tests with different treatments

| Treatment and test (weeks after STZ) | Object | Place |
|---|---|---|
| CSF (2) | 0.342 ± 0.09** | 0.292 ± 0.08** |
| CSF (4) | 0.397 ± 0.05** | 0.274 ± 0.06** |
| “ “ two days later | 0.339 ± 0.06** | NT |
| STZ(2) | 0.281 ± 0.11* | 0.231 ± 0.08* |
| STZ(4) | −0.175 ± 0.07^{#} | 0.000 ± 0.08^{#} |
| STZ(4) + ladostigil 1 mg/kg chronic | 0.238 ± 0.05** | 0.211 ± 0.07* |
| STZ(4) + ladostigil 1 mg/kg acute | 0.010 ± 0.08^{#} | NT |
| STZ(4) + 17 mg/kg acute | 0.348 ± 0.112** | NT |
| “ “ two days later | −0.112 ± 0.08^{#} | NT |

Data represents the mean ± s.e.m.
^{§}Discrimination index = time to explore new object − time to explore familiar object/total exploration time of both objects
^{‡}time to explore new position ob object − time to explore original position of object/total exploration time of both objects.
NT = not tested.
Significantly different from 0,
**P < 0.01,
*<0.05; significantly different from CSF,
^{#}P < 0.01

There were no differences between the groups in the total time spent by the rats exploring both objects in the discrimination phase of the object and place recognition tests. However, rats given icv CSF or icv STZ with ladostigil showed a significant discrimination index in both tests (significantly>0, P<0.05), while those given icv STZ alone failed to do so. This indicates that ladostigil prevents the impairment of episodic and spatial memory induced by icv STZ.

Ladostigil had no effect on the deficit in episodic memory when given at a single dose of 1 mg/kg, four weeks after icv injection of STZ. However, when increased to 17 mg/kg the memory deficit was abolished but returned two days later when ChE was no longer inhibited.

Spatial Memory Test

Repeated measures ANOVA for trial 1 revealed a significant effect of DAY (F_{4, 26}=25.67, P<0.0001) but no significant GROUP× DAY interaction. Post hoc test for means for groups over 5 days showed that rats given icv STZ only had significantly higher latencies than those injected with CSF or STZ and treated with ladostigil. In trial 2 there was also a highly significant effect of DAY (F_{4, 26}=23.0, P<0.0001), but no DAY× GROUP interaction (FIG. 6). There were also no significant differences between trial 1 and trial 2 in the escape latencies for any day in the different groups of rats, indicating that icv-STZ did not significantly impair working memory in this test.

Discussion

Icv injection of STZ in rats, which has been proposed as a model of the early pathophysiological changes in AD (Arnaiz et al, Impaired cerebral glucose metabolism and cognitive functioning predict deterioration in mild cognitive impairment, Neuroreport (2001) 12:851-5), induces reactive gliosis and oxidative-nitrative stress before the induction of memory deficits and pretreatment with ladostigil can prevent these effects. The reactive gliosis involves both microglia and astrocytes in the cingulate and motor cortex close to the site of cannula penetration, CA1 region of the hippocampus, and in the corpus callosum, medial and lateral septum close to the lateral ventrical. Evidence of oxidative nitrative stress is seen as NT immunoreactivity in astrocytes in the vicinity of cannula penetration, the CA1 area of the hippocampus and regions bordering the lateral ventricles. This is followed 2-5 weeks later by impairment of episodic and spatial memory, although no signs of neuronal damage or reduction in specific cholinergic markers in the cortex or hippocampus are present.

Pretreatment for a week before and one week after icv injection of STZ with ladostigil (1 mg/kg), a novel bifunctional ChE and MAO inhibitor that protects against cytotoxicity induced by a NO donor in cell culture, prevents the microglial activation and loss of astrocytes in the vicinity of the cannula penetration and hippocampal areas and prevents the increase in NT immunoreactivity. Development of memory deficits was prevented three weeks later.

Microglia are very sensitive to changes in their microenvironment and are readily activated in response to infection, brain injury and when the blood brain barrier is breached (McGeer et al., Inflammatory processes in Alzheimer's disease, Prog. Neuro-Psychopharmacol. Biol. Psychiatry (2003) 27:741-9). It was shown that icv injection of STZ greatly increased the number of activated microglia, characterized by shortening of their cellular processes and enlargement of soma, in the vicinity of the cannula penetration site, in the hippocampus and in regions bordering the lateral ventricles. The areas in the cortex and hippocampus showing the highest concentration of activated microglia had a much lower density of astrocytes than in CSF-injected rats. Their disappearance could have been caused by the secretion by microglia of proinflammatory and neurotoxic cytokines, NO and superoxide which are also deleterious to neurons(McCarty et al. 2006). Since astrocytes are important for sequestering glutamate, their loss may have resulted in excess glutamate activity and excitotoxic stress(Lipton and Rosenberg, 1994). In support of this suggestion it has been shown that the glutamate antagonist, memantine reduced the activation of microglia and astrocytes and the memory deficits seen after icv injection of Aβ₍₁₋₄₀₎ in rats (Miguel-Hidalgo et al., Neuroprotection by memantine against neurodegeneration induced by beta-amyloid (1-40), Brain Res. (2002) 958:210-21).

In an adjacent cortical penetration zone the predominant form of microglia had polymorphic shaped soma with a variety of fibrous processes. These contained a large number of activated astrocytes with long fibrous processes that showed increased GFAP immunoreactivity reminiscent of those induced by ROS and in aging rats (Finch, Neurons, glia, and plasticity in normal brain aging, Neurobiol. Aging (2003) 24:S123-7). The microglia in this zone did not differ in density from those in CSF-injected rats, and suggested that they stimulated the production of GFAP but did not release of cytokines that were toxic to astrocytes.

Activated astrocytes were also seen in different regions of the hippocampus of STZ-injected rats and contained significantly greater amounts of NT than after icv CSF. This may have resulted from the induction of ROS by STZ, like that seen in the pancreas after its parenteral administration (Takasu et al., 1991) and is also seen in aging rodents and human subjects. The NO and superoxide has been shown to alter mitochondrial function and cellular energy in astrocytes and in neighboring neurons (Bolanos et al., Regulation of glucose metabolism by nitrosative stress in neural cells, Mol. Aspects Med. (2004) 25: 61-73). Similar changes including increased NT immunoreactivity and depressed mitochondrial function are seen in the brains of subjects with AD (Finch, 2003). This could explain the alteration in glucose metabolism induced by icv STZ previously described (Nitsch et al., The intracerebroventricularly streptozotocin-treated rat: impairment of cerebral glucose metabolism resembles the alterations of carbohydrate metabolism of the brain in Alzheimer's disease, J. Neural Transm. (1989) 1:109-10). The increase in GFAP expression in activated astrocytes may also contribute to a reduction in synaptic function, ultimately affecting spatial and episodic memory. The presence of gliosis associated with oxidative-nitrative stress has been reported in AD (Griffin et al., Glial-neuronal interactions in Alzheimer's disease: the potential role of a ‘cytokine cycle’ in disease progression, Brain Pathol. (1998) 8:65-72) and could result in a suppression of mitochondrial function. Increased GFAP expression in activated astrocytes has been associated with a reduction in synaptic function (Finch, 2003). Thus, the increase in GFAP seen after icv STZ could have contributed to the impairment of spatial (place recognition) and episodic memory (object recognition test) without causing a measurable loss of cholinergic markers. Partial isolation of the cingulate cortex (situated between the bilateral cannulae) induced by icv STZ could also explain the deficit in episodic memory, since the glial scar formed around the cannula penetration site included all cortical layers and the corpus callosum. This scar could disrupt connections of the cingulate cortex to the peri-rhinal cortex which is important for objection recognition (Winters et al., 2004). The constellation of microglial activation, changes in astrocyte morphology and oxidative-nitrative stress that precede the appearance of deficits in glucose metabolism and memory supports the validity of the STZ icv injected rat as a model of the early pathophysiological changes in AD.

Although ladostigil is not an antioxidant, low concentrations have been shown to prevent cytotoxicity induced by ROS and NO in cultured neuronal cells (Youdim and Weinstock, 2001). In the present study, chronic administration of a low dose of ladostigil before and after STZ injection significantly reduced the alterations in microglia and astrocytes prevented the increase in NT immunoreactivity, and three weeks later, the development of episodic memory deficits. It is possible that the prevention by ladostigil of the loss of object recognition resulted from ChE inhibition of 28% in the cortex. However, this cannot explain its effect on place recognition, which depends on hippocampal cholinergic activity and in which no ChE inhibition occurred. It is therefore likely that the neuroprotective effect of ladostigil resulted from other actions of the drug. These could include a direct effect on microglia as shown in preliminary data from experiments in our laboratory in which concentrations of 0.1-10 μM reduced by 20-40% the release of NO from cultured microglia induced by LPS. These concentrations are too low to inhibit ChE in microglia. Ladostigil (0.1-10 μM) also prevented apoptosis in dopaminergic neuroblastoma SH-SY5Y cells exposed to the NO donor Sin1, by reducing 14 the fall in the mitochondrial membrane potential (Maruyama et al., 2003). This action probably does not result from MAO or ChE inhibition since it is shared by other propargylamine-containing aminoindan derivatives that have no effect on these enzymes.

Chronic once daily treatment with ladostigil (1 mg/kg) reduced the number of activated microglia and restored astrocytes in zone (i) of the cannula penetration site after icv STZ injection to those seen after injection of CSF. It also prevented the increase in NT immunoreactivity and development of memory deficits thereby providing an etiological connection between them. The mechanism by which ladostigil produces these anti-inflammatory and neuroprotective effects is not fully understood. One possibility is that it activates α7 nicotinic receptors indirectly as a result of AChE inhibition (Shytle et al., Cholinergic modulation of microglial activation by alpha 7 nicotinic receptors, J. Neurochem. (2004) 89:337-43). Although such an action could possibly contribute to the prevention of gliosis in the cortex in which it produced about a 20% inhibition of AChE, it cannot explain the marked reduction in NT immunoreactivity and astrocyte activation in the hippocampus in which no significant AChE inhibition occurred. Neither does the neuroprotection result from MAO-B inhibition which did not exceed 15%. A more likely explanation is that ladostigil reduces apoptosis and cell death in both neurons and astrocytes by preventing the fall the mitochondrial membrane potential induced by NO and superoxide anion released from microglia (Takuma et al., Astrocyte apoptosis: implications for neuroprotection, Prog. Neurobiol. (2004) 72:111-127). Such an action of ladostigil was demonstrated in dopaminergic neuroblastoma SH-SY5Y cells exposed to the NO donor, Sin1.

Acute administration of ladostigil (1 mg/kg) that only inhibited ChE by about 8% was unable to reverse the memory deficits once they had occurred. This was achieved by a larger dose that inhibited cortical ChE by 40%. The data suggest that prevention of gliosis, oxidative-nitrative stress and memory deficits induced by chronic administration of a low dose of ladostigil results from a combination of actions on neuronal and glial cells. The ability to inhibit ChE may contribute to the effect on episodic memory.

### EXAMPLE 3

The clinical condition of mild cognitive impairment (MCI) is one in which persons experience memory loss which is greater than what would be expected for their age (Petersen et al., 2001). A significant proportion of subjects with MCI develop dementia of the Alzheimer type (AD) within a few years. ChE inhibitors slow the progression of cognitive impairment in AD, but it is not yet known whether they can delay or prevent the progression of MCI to AD. Like humans, rats develop an age-related memory loss that is associated with a reduction in the number and size of cholinergic nerve terminals on cortical pyramidal cells (Turrini et al., 2001, Casu et al., 2002). At the age of 18 months, rats already show a 40-50% reduction in ACh release in the cortex and hippocampus. This can be increased to normal levels by acute administration of ChE inhibitors in doses that improve their memory deficits (Scali et al., 2002). However, it is not known whether any ChE inhibitor, and ladostigil in particular, can prevent the progression to memory impairment in aged rats; nor is it known what dosage would be effective.

Experiments were performed in 30 male rats of the Wistar strain. At the age of 16 months 80% of them showed intact object recognition, compared to 90% of those aged 3 months. The rats were housed in pairs at an ambient temperature of 21° C. They were weighed once weekly and their fluid intake measured daily for two weeks and then twice weekly for the remaining 4 months. Ten of the rats were started on ladostigil 1 mg/kg/day in the drinking fluid when aged 16 months and given the drug for four months. The remaining 10 rats of the same age were given regular drinking water. At the end of the four months spatial memory of these 20 rats and of another 10 aged 3 months was tested in the Morris water maze as previously described (Shoham et al, 2006).

The aged untreated rats showed a significantly slower acquisition of spatial learning than the young rats. The difference in learning between young and old rats was substantially abolished by low dose ladostigil treatment (FIG. 1a, b). The data show that chronic treatment with a low dose of ladostigil can prevent the development of memory deficits in aged rats.

Discussion

Mild cognitive impairment (MCI) is a condition generally characterized by mild recent memory loss without dementia or significant impairment of other cognitive functions. MCI causes memory problems that are greater than normally expected with aging, but a person with MCI does not show other symptoms of dementia, such as impaired judgment or reasoning. The causes of MCI are not well understood. An advisory panel to the US Food and Drug Administration ruled in 2001 that MCI, a condition separate from Alzheimer's disease, is a valid target for new drug therapies. The Peripheral and Central Nervous System Drugs Advisory Committee has stated that more than 80% of patients with mild cognitive impairment develop Alzheimer's disease within 10 years. Scientists are still working to understand MCI and its relationship to Alzheimer's disease. Because basic questions about this disorder remain to be answered, the definition of MCI continues to evolve. (Mild Cognitive Impairment-Alzheimer's Part XVI, Harold Rubin, MS, ABD, CRC, Nov. 14, 2006).

Mild cognitive impairment has recently been shown to exist in two distinct subtypes: neurodegenerative MCI and vascular MCI. Subjects with neurodegenerative MCI exhibit medial temporal lobe atrophy, while subjects with vascular MCI present with vascular lesions, both of which be observed by magnetic resonance imaging (MRI). It has recently been reported that neurodegenerative MCI subjects possess impaired cognitive domains involving memory, which is found to be predictive for conversion to dementia or Alzheimer's disease (Meyer et al., 2005). Until recently, little evidence to support the hypothesis that MCI was a precursor for Alzheimer's existed; however, Meyer et al. clearly teach that MCI can take the form of a neurodegenerative disorder and later take the form of dementia or Alzheimer's.

What is claimed is:

1. A method of preventing the appearance of symptoms of a neurodegenerative disease in a subject predisposed to the neurodegenerative disease, or of slowing the progression of an early stage neurodegenerative disease to a more advanced stage of the neurodegenerative disease in an afflicted subject, comprising administering to the subject an amount of R(+)-6-(N-methyl,N-ethyl-carbamoyloxy)-N′-propargyl-1-aminoindan or a salt thereof effective to prevent the appearance of symptoms of the disease or to slow the progression of the disease

2. The method of claim 1, wherein the effective amount administered to the subject is a less than cholinesterase-inhibitory amount or a less than monoamine oxidase-inhibitory amount of R(+)-6-(N-methyl,N-ethyl-carbamoyloxy)-N′-propargyl-1-aminoindan or a salt thereof.

3. The method of claim 2, comprising administering to the subject a less than cholinesterase-inhibitory amount and a less than monoamine oxidase-inhibitory amount of R(+)-6-(N-methyl,N-ethyl-carbamoyloxy)-N′-propargyl-1-aminoindan or a salt thereof.

4. The method of any of claims 1-3, comprising administration of a salt of R(+)-6-(N-methyl,N-ethyl-carbamoyloxy)-N′-propargyl-1-aminoindan.

5. The method of claim 4, wherein the salt of R(+)-6-(N-methyl,N-ethyl-carbamoyloxy)-N′-propargyl-1-aminoindan is the tartrate salt.

6. The method of any one of claims 1-5, wherein the subject is a human.

7. The method of claim 6, wherein the amount of R(+)-6-(N-methyl,N-ethyl-carbamoyloxy)-N′-propargyl-1-aminoindan is no more than 0.37 mg/kg/day of the subject, or a corresponding amount of the salt thereof.

8. The method of claim 7, wherein the salt is the tartrate salt and the corresponding amount of the salt is no more than 0.46 mg/kg/day of the subject.

9. The method of claim 6, wherein the amount of R(+)-6-(N-methyl,N-ethyl-carbamoyloxy)-N′-propargyl-1-aminoindan is no more than 2.3 mg/kg/day of the subject, or a corresponding amount of the salt thereof.

10. The method of claim 9, wherein the salt is the tartrate salt and the corresponding amount of the salt is no more than 2.9 mg/kg/day of the subject.

11. The method of claim 6, wherein the amount of R(+)-6-(N-methyl,N-ethyl-carbamoyloxy)-N′-propargyl-1-aminoindan or a salt thereof administered is 10 mg/day-25 mg/day.

12. The method of claim 6, wherein the amount of R(+)-6-(N-methyl,N-ethyl-carbamoyloxy)-N′-propargyl-1-aminoindan or a salt thereof administered is 0.14 mg/kg/day-0.42 mg/kg/day of the subject.

13. The method of claim 12, wherein the amount of R(+)-6-(N-methyl,N-ethyl-carbamoyloxy)-N′-propargyl-1-aminoindan or a salt thereof administered is 0.15 mg/kg/day of the subject.

14. The method of any of claims 1-13, further comprising administering to the subject an antioxidant.

15. The method of any of claims 1-14, wherein the neurodegenerative disease is Alzheimer's disease, dementia, mild cognitive impairment, Parkinson's disease, age-related macular degeneration, or amyotrophic lateral sclerosis.

16. The method of claim 15, wherein the disease is dementia and the dementia is static dementia, senile dementia, presenile dementia, progressive dementia, vascular dementia or Lewy body dementia.

17. The method of any of claims 1-16, wherein the method slows the progression of an early stage neurodegenerative disease to a more advanced stage of the neurodegenerative disease in the subject.

18. The method of any of claims 1-16, wherein the method prevents the appearance of symptoms of a neurodegenerative disease in a subject predisposed to the disease.

19. A method of reducing oxidative stress in the brain of a subject afflicted with oxidative stress, comprising administering to the subject a less than cholinesterase-inhibitory amount or a less than monoamine oxidase-inhibitory amount of R(+)-6-(N-methyl,N-ethyl-carbamoyloxy)-N′-propargyl-1-aminoindan or a salt thereof effective to reduce oxidative stress in the brain of the subject.

20. The method of claim 19, comprising administering to the subject a less than cholinesterase-inhibitory amount and a less than monoamine oxidase-inhibitory amount of R(+)-6-(N-methyl,N-ethyl-carbamoyloxy)-N′-propargyl-1-aminoindan or a salt thereof effective to treat the subject.

21. The method of claim 19 or 20, comprising administration of a salt of R(+)-6-(N-methyl,N-ethyl-carbamoyloxy)-N′-propargyl-1-aminoindan.

22. The method of claim 21, wherein the salt of R(+)-6-(N-methyl,N-ethyl-carbamoyloxy)-N′-propargyl-1-aminoindan is the tartrate salt.

23. The method of any of claims 19-22, wherein the subject is a human.

24. The method of claim 23, wherein the less than monoamine oxidase-inhibitory amount of R(+)-6-(N-methyl,N-ethyl-carbamoyloxy)-N′-propargyl-1-aminoindan is no more than 0.37 mg/kg/day of the subject, or a corresponding amount of the salt thereof.

25. The method of claim 24, wherein the salt is the tartrate salt and the corresponding amount of the salt is no more than 0.46 mg/kg/day of the subject.

26. The method of claim 23, wherein the less than cholinesterase-inhibitory amount of R(+)-6-(N-methyl,N-ethyl-carbamoyloxy)-N′-propargyl-1-aminoindan is no more than 2.3 mg/kg/day of the subject, or a corresponding amount of the salt thereof.

27. The method of claim 26, wherein the salt is the tartrate salt and the corresponding amount of the salt is no more than 2.9 mg/kg/day of the subject.

28. The method of claims 23, wherein the amount of R(+)-6-(N-methyl,N-ethyl-carbamoyloxy)-N′-propargyl-1-aminoindan or a salt thereof administered is 10 mg/day-25 mg/day of the subject.

29. The method of any of claim 19-28, wherein the oxidative stress occurs in the hippocampus region of the brain.

30. The method of claim 29, wherein the oxidative stress occurs in the astrocytes of the hippocampus region of the brain.

31. A method of treating a subject afflicted with mild cognitive impairment comprising administering to the subject an amount of R(+)-6-(N-methyl,N-ethyl-carbamoyloxy)-N′-propargyl-1-aminoindan or a salt thereof effective to treat the subject.

32. The method of claim 31, wherein the effective amount is a less than cholinesterase-inhibitory amount or a less than monoamine oxidase-inhibitory amount of R(+)-6-(N-methyl,N-ethyl-carbamoyloxy)-N′-propargyl-1-aminoindan or a salt thereof effective to treat the subject.

33. The method of claim 32, wherein the administration to the subject of the less than cholinesterase-inhibitory amount or the less than monoamine oxidase-inhibitory amount of R(+)-6-(N-methyl,N-ethyl-carbamoyloxy)-N′-propargyl-1-aminoindan or the salt thereof is effective to delay the progress of MCI to Alzheimer's disease.

34. The method of any of claims 31-33, comprising administering to the subject a less than cholinesterase-inhibitory amount and less than monoamine oxidase-inhibitory amount of R(+)-6-(N-methyl,N-ethyl-carbamoyloxy)-N′-propargyl-1-aminoindan or a salt thereof effective to treat the subject.

35. The method of any one of claims 31-34, comprising administration of a salt of R(+)-6-(N-methyl,N-ethyl-carbamoyloxy)-N′-propargyl-1-aminoindan.

36. The method of claim 35, wherein the salt of R(+)-6-(N-methyl,N-ethyl-carbamoyloxy)-N′-propargyl-1-aminoindan is the tartrate salt.

37. The method of any of claims 31-36, wherein the subject is a human.

38. The method of claim 37, wherein the amount of R(+)-6-(N-methyl,N-ethyl-carbamoyloxy)-N′-propargyl-1-aminoindan is no more than 0.37 mg/kg/day of the subject, or a corresponding amount of the salt thereof.

39. The method of claim 38, wherein the salt is the tartrate salt and the corresponding amount of the salt is no more than 0.46 mg/kg/day of the subject.

40. The method of claim 37, wherein the amount of R(+)-6-(N-methyl,N-ethyl-carbamoyloxy)-N′-propargyl-1-aminoindan is no more than 2.3 mg/kg/day of the subject, or a corresponding amount of the salt thereof.

41. The method of claim 40, wherein the salt is the tartrate salt and the corresponding amount of the salt is no more than 2.9 mg/kg/day of the subject.

42. The method of claim 37, wherein the amount of R(+)-6-(N-methyl,N-ethyl-carbamoyloxy)-N′-propargyl-1-aminoindan or a salt thereof administered is 10 mg/day-25 mg/day of the subject.

43. A pharmaceutical composition in unit dosage form comprising R(+)-6-(N-methyl,N-ethyl-carbamoyloxy)-N′-propargyl-1-aminoindan in an amount of up to 25 mg, or a corresponding amount of a pharmaceutically acceptable salt thereof, and a pharmaceutically acceptable carrier.

44. The pharmaceutical composition of claim 43, comprising R(+)-6-(N-methyl,N-ethyl-carbamoyloxy)-N′-propargyl-1-aminoindan in an amount of 10 mg-25 mg or a corresponding amount of a pharmaceutically acceptable salt thereof, and a pharmaceutically acceptable carrier.

45. The pharmaceutical composition of claim 43 or 44, wherein the salt is the tartrate salt.
